(19) United States
(12) Patent Application Publication
(10) Pub. No.: US 2004/0254468 A1
(43) Pub. Date: Dec. 16, 2004

(54) Mapping and tracking blood flow using reduced-element probe

(75) Inventors: Donald Herzog, Collingswood, NJ (US); Kenneth Abend, Huntingdon Valley, PA (US)

Correspondence Address: John K. Kim, McCarter & English, LLP, Four Gateway Center, 100 Mulberry Street, Newark, NJ, 07102, US

(73) Assignee: Vuesonix Sensors, Inc.

(21) Appl. No.: 10/810,322

(22) Filed: Mar. 26, 2004

Related U.S. Application Data
(60) Provisional application No. 60/458,197, filed on Mar. 27, 2003.

(51) Int. Cl. A61B 8/06

(52) U.S. Cl. 600/453

(57) ABSTRACT

A 1 ½ D probe is used in acoustic Doppler blood flow imaging to accurately determine the position of blood vessel in three dimensions. The 1 ½ D probe has closely spaced elements in the x direction and widely spaced elements in the y direction. Doppler power measurements are used to determine the y position of the blood vessel to an accuracy better than achieved by prior art techniques.

### PRIORITY

This application claims the benefit of U.S. Provisional Application No. 60/458,197 filed Mar. 27, 2003.

### BACKGROUND OF THE INVENTION

1. Field of the Invention

The invention is in the field of ultrasound imaging, primarily for medical purposes.

2. Brief Description of the Background Art

The technology that serves as a basis for the herein disclosed invention is disclosed and claimed in U.S. Pat. No. 6,682,483 B1, issued Jan. 27, 2004 (the '483 patent) and U.S. Pat. No. 6,524,253 B1, issued Feb. 25, 2003 (the '253 patent), the disclosures of which are incorporated by reference, in their entirety herein.

Both patents explicitly disclose probes with one- and two-dimensional arrays of ultrasonic transducer elements that may be thinned. While most ultrasound phased array probes currently in use are not thinned, ultrasound probes do exist that have a small number of rows, each of which is a phased array. Such probes are called 1½-D devices. In one direction (a row) the elements are placed no more than a wavelength apart so that the array's transmit and receive beams may be steered and/or dynamically focused in that direction. In the other direction the. elements are longer than a wavelength and are consequently spaced more than a wavelength apart in that direction, limiting the amount of beam steering that can be accomplished. The beam steering and dynamic focusing is accomplished by phase control of the transmitted pulses and digital analysis of the received reflections. The array of elements is filled in the sense that there are no significant gaps between the elements. Such a probe, permitting only limited beam steering in one direction is disclosed. The purpose of this invention is to show how the technology of the above cited patents can be applied to such a 1½-D probe using a novel technique.

### SUMMARY OF THE INVENTION

The methods described in the above cited patents for determining parameters of blood flow, for mapping and tracking the flow, and for volumetric imaging, are applicable to a variety of sensor arrays. The array could be one-dimensional or two-dimensional. In the '483 patent the elements can be closely spaced (to permit steering and focusing without grating lobes) or they may be spaced farther apart. One- and two-dimensional arrays were cited as examples in both patents. In particular, the simple rectangular two-dimensional array configuration of the '483 patent was described in detail for the case where both the rows and the columns are more than ½- to 1 wavelength apart. The herein disclosed invention discloses the simple 1½-D case, the case where only the rows are more than a wavelength apart, while the columns are not. An example of such a probe is disclosed in U.S. Pat. No. 6,238,346 (M. K. Mason of Agilent Technologies), the disclosure of which is hereby incorporated by reference herein. It is only a special case of the general configuration described in the '483 patent.

A 1½-D probe consists of several wide rows, where each row contains many closely spaced elements. The spacing between columns (i.e., in the row direction, the x direction) is on the order of a wavelength or less while the center to center spacing between the rows (i.e., along each column, the y direction) is on the order of several (e.g., 2 to 6) wavelengths. While only a section of the probe is active at a time, the active section encompasses far more columns than rows. The number of active rows is so small, that no electronic steering of the transmitted energy is currently attempted in elevation (the ‘y’direction). In prior art devices electronic steering and dynamic focusing is only done in azimuth (the ‘x’direction).

The inventions disclosed in the '483 and '253 patents, using a square or symmetric 2-D array (number of columns equals number of rows and column spacing equals row spacing), can be used in two different ways. If the number of elements is small, they can be used to map and track blood flow in 3-Dimensional space (as described in the '483 patent) and the results of this mapping is subsequently used to determine parameters of blood flow such as vector velocity and flow volume. If the number of elements (and hence the active aperture size) is large, such probes are used for volumetric or 3-D imaging (as described briefly in the '483 patent and in detail in the '253 patent, U.S. application. Ser. No. 10/327,265, filed on Dec. 20, 2002, and U.S. Provisional Application. Ser. No. 60/446,162, filed Feb. 10, 2003). In the latter case, mapping and tracking of blood flow (e.g., for use in determining parameters of blood flow) becomes relatively easy. It is only necessary to determine the centerline of the flow, which is the center of the 3-D power Doppler image. For example, the center may be approximated by finding either the mean or the median of y and of z (z=depth or range, determined by time delay) for each x in the power Doppler or color flow image. A more accurate computation refines the centerline location by examining slices perpendicular to the initial centerline (instead of slices at constant x).

The methods of the above cited references and U.S. Provisional Application No. 60/446,162 (the disclosure of which is hereby incorporated by reference herein) are directly applicable to a 1½-D array using the technique disclosed below. Here the y component of the centerline is determined using monopulse techniques (as that term is commonly used, for example, in RADAR applications), even though the active columns have too few elements to accurately position an image of the vessel in the y direction. The result is a map of the vessel centerline in three dimensions and a power Doppler and/or color flow image of the vessel in the x-z plane. The centerline provides a cue and direction for the operator as to how to position the probe. The centerline map, along with the measured color Doppler data is used to determine vector velocity. The cross-sectional area is inferred (approximately) from the planer image by assuming that the vessel cross-section is circular. This then allows for an estimate of flow volume. The ratio of minimum vector velocity (at the center line) to maximum vector velocity for a given vessel is all that is needed to determine percent stenosis.

Further features and advantages of the invention will appear more clearly on a reading of the following detailed description of exemplary embodiments of the invention, which are given below by way of example only with reference to the accompanying drawings.

### BRIEF DESCRIPTION OF THE DRAWINGS

FIG. 1 is a plan view of a section of an exemplary ultrasound probe having three rows of closely spaced transducer elements in the x-y plane.

FIG. 2 is a schematic representation of pixels in the x-z plane resolving the position of a blood vessel.

FIG. 3 is a plan view of pixels in the x-y plane produced by transmitting and detecting ultrasound energy using a probe of FIG. 1, with an unsteered pair of beams simultaneously formed in the y direction.

FIG. 4 is a plan view of pixels in the x-y plane produced as in FIG. 3, with modest steering of the beam pair in the y direction.

FIG. 5 is a block diagram of an exemplary embodiment of the analog and digital control, analysis, and user interface elements of an ultrasound blood flow monitoring and imaging system.

### DETAILED DESCRIPTION OF THE INVENTION

Using the technique disclosed below, the methods disclosed in the above cited reference can be used with a 1½-D array such as one described in the '346 patent or as illustrated in FIG. 1. FIG. 1 shows a portion of a probe 1 with three rows 2, 3, 4 of transducer elements. The elements are closely spaced along each row (the “x” direction) with a spacing of the order of an acoustic wave length or less. The elements of each column (see, for example, the cross hatched elements 5, 6, 7 of one column) are several wave lengths long. Each element is individually accessed by the control circuitry so that the probe 1 can be accessed a section at a time.

### Example of 3-D Blood-Flow Mapping and Parameter-Extraction for a 1½-D Array

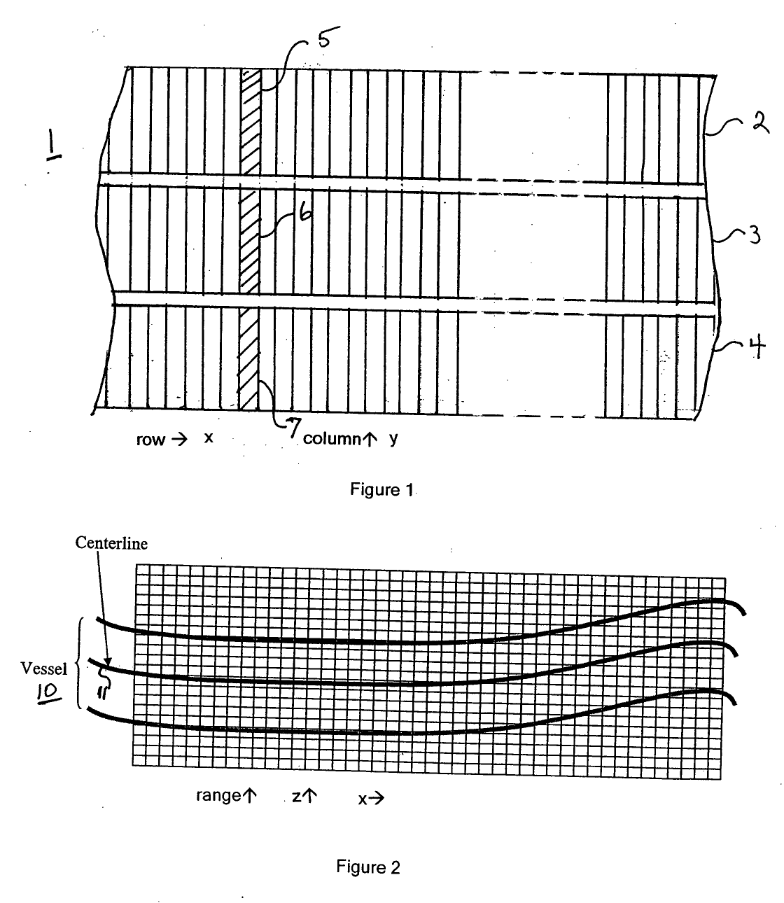

Consider a 384-element array consisting of 3 rows of 128 elements each, a portion of which is illustrated in FIG. 3. (Another example would be a 640-element array consisting of 5 such rows.) FIG. 2 shows the pixels in a conventional power Doppler image of a blood vessel 10 formed in the range (z i.e., the depth perpendicular to the x-y plane) and azimuth or row (x) directions, along with an estimate of the centerline 11 of that image created by the array. The estimate is accurately derived from the image in the x-z plane because of the high resolution attained in the x (azimuth) and z (range or depth) directions. Unfortunately, this centerline cannot be used to compute accurate vector velocity because its y component is missing (i.e., the component out of the plane of the paper). Also, there is no way to know if the probe is aligned with the direction of the vessel, and even if it is, there is no way to know that the slice shown is through the center of the vessel. Elevation (y) information is needed.

The 2-D arrays described in the '253 patent provide an accurate 3-D centerline because the resolution in y is equivalent or comparable to the resolution in x, and a respectable field of view is attainable in both directions. If the 1½-D array, with correspondingly fewer elements, were to be used in place of 2-D array with beam steering in the y direction, the resolution in y would not be very fine and the y field of view would be so small that it barely exceeds the resolution in the y direction. For example, if one were to transmit with a segment of the middle row 3 and receive with elements in all rows 2, 3, 4 or a portion of all three rows, many receive beams are formed in x and only an unsteered pair of beams simultaneously formed in y. Subsequently, a different segment of the middle row would transmit and another such set of receive beams would be created. This would form a quasi 3-dimensional image with many resolution cells in x and z but only a limited number, for example two, in y.

This situation is illustrated in FIG. 3, illustrating the blood vessel's position in the x-y plane. The x-y pixels highly resolve the x-axis but poorly resolve the y-axis, with only two pixels that are overlapping. The y position of the centerline, however, can be estimated by determining the relative weight (i.e., Doppler power) of the overlapping pixels in the y-direction determining the position of the blood vessel relative to the upper or lower row of elements. In the z-direction the centerline is calculated from FIG. 1 using time delay data. The Doppler shift frequency data is also used to determine flow velocity (v), which is used to resolve overlapping positional information between closely spaced blood vessels, the blood vessels typically carrying blood with different velocities. No y-axis steering of the simultaneously formed pair of beams is used. In the y direction a monopulse technique is used to accurately estimate the y component of each centerline pixel illustrated in FIG. 2 using the relative weight data. The centerline can then be accurately plotted as in FIG. 3, and imaged in three dimensions using voxels (three dimensional pixels) with small dimensions in y as well as in x and z. For probes with more than three rows, a larger, but limited, number of unsteered simultaneously formed beams are formed, extending the ability to accurately locate the y position of the blood vessel.

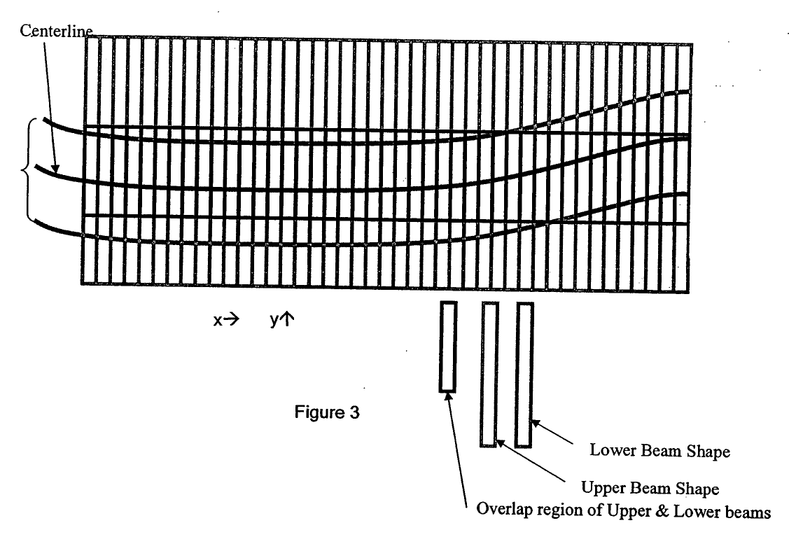

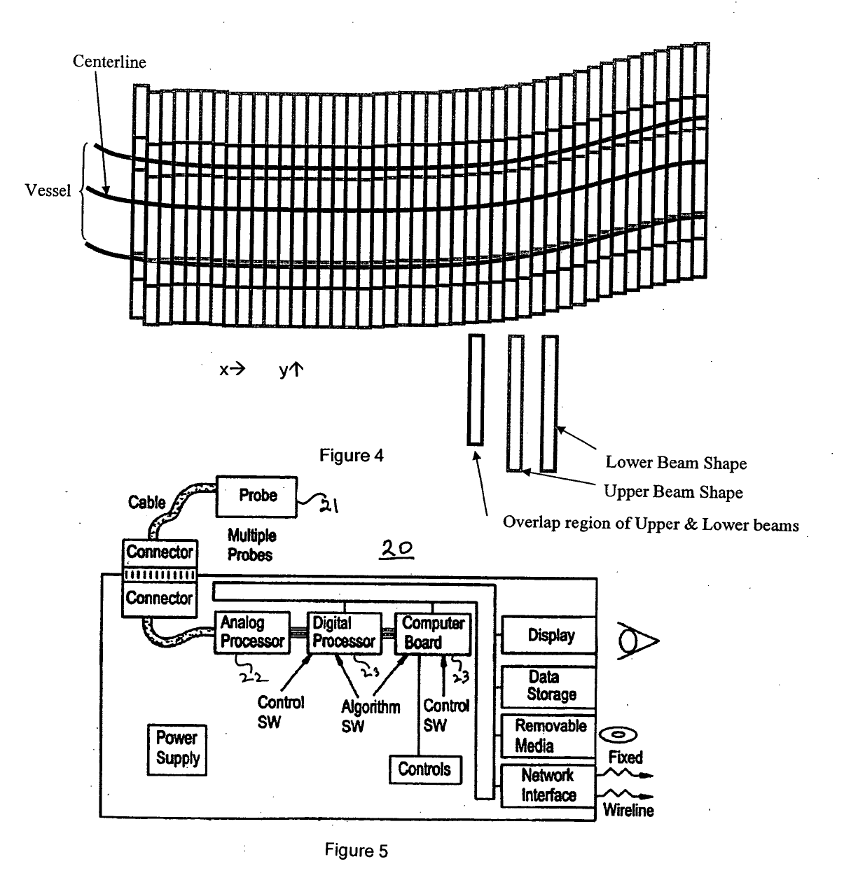

FIG. 4 is the same as FIG. 3 except the centerline can be determined more accurately by modestly steering the receive beams from each column of elements so that the Doppler power received from each of the upper and lower beams for each section of the vessel is equal (monopulse power difference=zero). This makes use of overlapping pixels in the y-direction from beams that are steered in unison. Standard monopulse tracking techniques, such as those used in radar systems, are used to drive the monopulse power difference to zero, the degree of steering at zero difference determining the y position of the blood vessel. In the z-direction the centerline is calculated using time delay data, as illustrated in FIG. 1.

FIG. 5 is a block diagram of an exemplary embodiment of the analog and digital control, analysis, and user interface elements of an ultrasound blood flow monitoring and imaging system 20. It shows a probe 21 feeding the analog 22 and digital 23 signal processing devices under software control, including an output module, and the display, storage and communication elements of the user interface. This equipment is more fully described in the '483 patent.

It will be understood that the embodiments described herein are merely exemplary and that a person skilled in the art may make many variations and modifications without departing from the spirit and scope of the invention. All such variations and modifications are intended to be included within the scope of the invention.

What is claimed is:

1. A method for locating the position of a blood vessel in a subject by receiving and analyzing Doppler shifted reflections of ultrasonic energy of wave lengths λ transmitted into the subject by a 1½D probe terminated by an array of transducer elements, the array comprising a plurality of rows, the element having center to center spacing on the order of λ or less along the rows and forming columns with center to center spacing of several times λ perpendicular to the rows, the method comprising:
a) transmitting pulses of ultrasonic energy into the subject;
b) receiving Doppler shifted reflections from the blood vessel;
c) analytically forming a limited number of simultaneously formed receive beams at each column position;
d) steering the receive beams in a first direction along the rows to accurately determine the row direction position of the blood vessel;
e) determining the blood vessel's position in a second direction along the columns at each column position by comparison of Doppler power detected by the limited number of receive beams; and
f) determining the blood vessels position in a direction perpendicular to both the rows and columns by time delay data.

2. A method of claim 1 comprising modestly steering the simultaneously formed receive beams in the second direction and determining a steering degree at each column position at which the Doppler power received by each beam is the same, the degree of steering producing zero power difference at each column position, thus determining the blood vessel's position in the second direction at each column position.

3. A method of claim 2 comprising determining a difference between the Doppler power received by each beam and driving the difference to zero by a monopulse technique.

4. A method of claim 1 in which the probe has three rows and the received ultrasound energy is analyzed to form a pair of simultaneously formed receive beams.

5. A method of claim 1 comprising transmitting the blood vessel's position to a user interface.

6. A method of claim 5 comprising displaying the blood vessel's position.

7. A method of claim 5 comprising storing the blood vessel's position.

8. A method of claim 1 comprising resolving the blood vessel's position from a closely spaced second blood vessel's position by comparing Doppler shift data from the blood vessel with Doppler shift data from the second blood vessel.

9. A device for locating the position of a blood vessel in a subject by receiving and analyzing Doppler shifted reflections of ultrasonic energy of wave lengths λ transmitted into the subject by a 1½D probe terminated by an array of transducer elements, the array comprising a plurality of rows, the element having center to center spacing on the order of λ or less along the rows and forming columns with center to center spacing of several times λ perpendicular to the rows, the device comprising:
a) an ultrasound pulse transmitter for energizing the 1½D probe to transmit pulses of ultrasound energy;
b) an analog processor for receiving ultrasound signals, from the probe reflected by the subject and detected by the probe and producing a digital data stream;
c) a digital processor under software control for analyzing the digital data stream to determine the blood vessel's position by analytically steering receive beams in the row direction at each column position to determine the row-direction position of the blood vessel, analyzing time delay information in the digital data stream to determine the range of the blood vessel, and analytically forming a limited number of simultaneously formed receive beams at each column position for determining the blood vessels position in the column direction by comparing Doppler power detected by the limited number of receive beams, the digital processor producing analyzed data;
d) an output module for processing the analyzed data and producing output signals for display or storage.

10. A device of claim 9 in which the digital processor is adapted for modestly steering the simultaneously formed receive beams in the second direction and determining a steering degree at each column position at which the Doppler power received each beam is the same, the degree of steering producing zero power difference at each column position determining the blood vessel's position in the column direction at each column position.

11. A device of claim 10 in which the digital processor is adapted for determining a difference between the Doppler power received by each beam and driving the difference to zero by a monopulse technique.

12. A device of claim 9 comprising the 1½D probe.

13. A device of claim 9 in which the 1½D probe has three rows and the digital processor is adapted to produce a pair of simultaneously formed receive beams.
